(19) United States
(12) Plant Patent Application Publication
(10) Pub. No.: US 2003/0093848 P1
(43) Pub. Date: May 15, 2003

(54) Begonia plant named 'Fuga'

(75) Inventor: Andre Smaal, Aalsmeerderweg (NL)

Correspondence Address: Russell D. Orkin, 700 Koppers Building, 436 Seventh Avenue, Pittsburgh, PA, 15219-1818, US

(73) Assignee: Begonia Breeders Association B.V.

(21) Appl. No.: 10/010,496

(22) Filed: Nov. 13, 2001

(51) Int. Cl. A01H 5/00

(52) U.S. Cl. PLT/348

(57) ABSTRACT

A new and distinct begonia plant with red-pink colored double flowers.

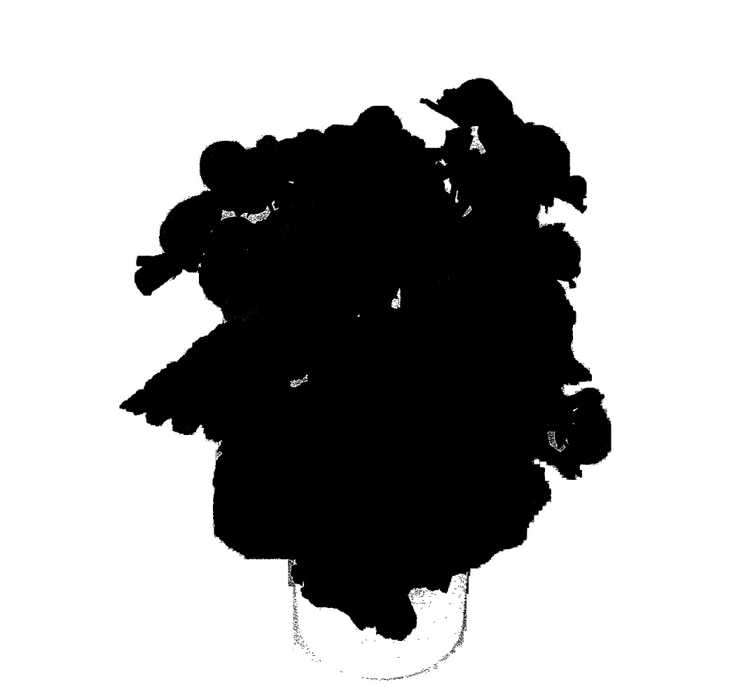

### BACKGROUND OF THE INVENTION

The present invention comprises a new and distinct cultivar of Begonia elatior known by the varietal name ‘Fuga’. The new variety was discovered in Aalsmeer, Netherlands, in 01998. The new variety is a product of a planned breeding program and is a cross an unnamed, unpatented variety of Begonia tuberosa (female) and an unnamed, unpatented variety of Begonia socotrana (male). The breeding program is directed to establishing various phenotype characteristics. The new variety differs in its flower color and its double flowers as compared to its parents. The new variety was first asexually reproduced by cuttings in Aalsmeer in February of 2000. The new variety has been trial and field tested and has been found to retain its distinctive characteristics and remain true to type through successive propagations.

The new variety, when grown in a glass greenhouse has a response time for rooting from a terminal cutting of 14 days at 23° C.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions by cuttings in a controlled greenhouse environment. Color references are primarily to the R.H.S. Colour Chart of The Royal Horticultural Society of London.

### PLANT

Classification:

Botanical.—Begonia elatior.

Commercial.—Begonia.

Form: Upright.

Size: About 40 cm tall from top of media surface; 35 cm wide in 15 cm container 10 weeks after planting, with pinching.

Growth: Moderate.

Time to initiate roots: About 14 days at 22° C.

Time to develop roots: About 18 days at 22° C.

Rooting habit: Fine, fibrous and well-branched

Lateral branches:

Appearance/aspect.—Cylindrical; upright to somewhat outwardly angled.

Strength.—Very good.

Length.—About 19 cm.

Diameter.—About 1 cm.

Internode length.—About 3.8 cm.

Texture.—Mostly smooth with sparse small white hairs; fleshy.

Leaves:

Arrangement.—Alternate.

Shape.—Rounded with acute apex and cordate base with overlap.

Margin.—Doubly-serrated; bi-crenate.

Size.—15 cm wide; 20 cm long.

Quantity.—About 8 leaves per lateral branch.

Stipules.—2 per leaf sized 7 mm×7 mm.

Texture.—Smooth; shiny and leathery.

Color: Upper surface: Green Group 139A. Lower surface: Green Group 138A with a blush of Red Group 50B.

Venation.—Palmate; Green Group 157A on upper surface.

Petiole.—About 5 cm long; 5 mm in diameter.

### FLOWERS

Habit: Continuous flowering.

Size: 6 cm in diameter; 1 cm deep.

Tepals:

Arrangement.—Double; 14-20 tepals per flower.

Size of largest tepal.—2 cm long; 3 cm wide.

Shape.—Oval.

Margin.—Entire.

Color:

Upper surface.—Red Group 48B.

Lower surface.—Red Group 48C.

Flower buds: About 2 cm long (in autumn); about 1.5 cm wide.

Peduncles:

Angle.—Mostly erect.

Strength.—Strong but flexible.

Length.—About 4.5 cm.

Color.—Yellow-Green Group 146D.

Pedicels:

Angle.—About 30-45° from vertical.

Strength.—Strong but flexible.

Length.—About 1.2 cm.

Color.—Yellow-Green Group 146D with blush of Red Group 39B.

Bracts:

Arrangement.—Two; opposite.

Shape.—Very broadly cordate; acute apex.

Margin.—Entire.

Color.—Light Green.

### REPRODUCTIVE ORGANS

Stamens: None observed.

Pistils: None observed.

Postproduction longevity:

Flowers.—About 5-6 weeks.

Plants.—About 8-9 weeks.

Disease resistance: Mildew tolerant.

Seed Production: Seed production has not been observed as reproductive organs are not formed.

I claim:

1. A new and distinct variety of begonia plant substantially as shown and described.
